(19) United States
(12) Patent Application Publication
(10) Pub. No.: US 2018/0055953 A1
(43) Pub. Date: Mar. 1, 2018

(54) ATHEROSCLEROSIS IMAGING AGENTS AND METHODS OF USING THE SAME

(71) Applicant: THE GENERAL HOSPITAL CORPORATION, Boston, MA (US)

(72) Inventors: Farouc A. Jaffer, Jamaica Plain, MA (US); Adam Mauskapf, Boston, MA (US); Eric Osborn, Boston, MA (US); Guillermo J. Tearney, Boston, MA (US)

(21) Appl. No.: 15/556,046

(22) PCT Filed: Mar. 7, 2016
(86) PCT No.: PCT/US16/21198

§ 371 Date: Sep. 6, 2017

Related U.S. Application Data
(60) Provisional application No. 62/129,243, filed on Mar. 6, 2015.

(51) Int. Cl. A61K 49/00; A61B 5/00

(52) U.S. Cl. CPC A61K 49/006; A61B 5/004; A61K 49/003; A61B 5/0059

(57) ABSTRACT

Methods for detecting the presence of atherosclerotic structures in order to diagnose or prevent atherosclerosis are provided herein. In particular, it has been found that methylene blue injected intravenously acts as an excellent indicator because the compound targets high-risk plaque, atheroma, macrophages, and other atherosclerotic structures formed within the endothelial walls of a vessel of a subject. Because the compound provides a unique binding profile with uptake only in plaque or atheroma, and not the normal or healthy vascular interstitial tissue, methylene blue maintains a good plaque-to-background ratio for imaging purposes. This enables healthcare providers to determine the status of atherosclerosis development in vivo within a patient with higher certainty and at lower costs. The disclosed methods allow for high-resolution mapping of plaque build-up, plaque pathobiology, and other atherosclerotic structures within a vessel of a subject by using methylene blue as an imaging agent.

### CROSS-REFERENCES TO RELATED APPLICATIONS

This application claims priority from U.S. Patent Application No. 62/129,243 filed Mar. 6, 2015.

### STATEMENT REGARDING FEDERALLY SPONSORED RESEARCH

Not Applicable.

### BACKGROUND OF THE INVENTION

### 1. Field of the Invention

The present disclosure relates to a method for the targeted imaging and detection of characteristic structures or molecules indicative of atherosclerosis. In particular, intravenously injected methylene blue or derivatives thereof may be used as imaging agents targeting atheroma or related structures in the vascular system of a subject.

### 2. Description of the Related Art

Atherosclerosis is a vascular or chronic inflammatory disease associated with the development of atherosclerotic plaque or atheroma, made up of macrophages and lipids, within vessel walls. This build-up of lesions in the arteries and veins of a patient may lead to clinical events such as myocardial infarction or stroke. Atheroma in veins or arteries of patients with developing atherosclerosis are primarily formed of macrophages and lipids. The macrophages or white blood cells in atheroma typically have taken up oxidized low-density lipoproteins (LDLs). The characteristics or structures related to atherosclerosis and thus used for diagnosis include the permeability of endothelial tissue and atherosclerotic plaques, atherogenesis or the formation of neovessels in plaque, plaque neovascularization, apoptosis of lipids or plaque, interplaque hemorrhage, plaque rupture, inflammation, a lipid-rich pool, lack of thick fibrous cap, insudation of plasma proteins, lack of extracellular matrix, albumin, alpha-globulins, microvessel growth, stenosis or lumen restriction, and intimal medial thickness.

Endothelial permeability is often targeted because imaging agents can accumulate at sites of inflammation due to this enhanced permeability. Normal endothelial walls in the vascular system have tight junctions without gaps or permeability. The abnormal endothelial lining, or intima, with this enhanced permeability including gaps allows extravasation of inflammatory cells and lipids, causing further plaque buildup. Moreover, hypoxia within atherosclerotic lesions cause the microvessels, or vasa vasorum, to undergo disorganized angiogenesis, or expansion outside of the vessel's normal state through neovascularization.

Angiography, which images the entire vessel lumen but not the vessel wall, has historically been used to suggest the diagnosis of atherosclerosis. Areas of neovascularization, microvessel growth, endothelial permeability, and others may be indicated by various targeting agents using angiography. Magnetic resonance (MR) angiography may be used in carotid arteries and aorta to show the accumulation of carotid plaque macrophages. However, MR angiograms using gadolinium contrast agents are inadequate to show atheroma or plaque in the smaller vessels of patients such as coronary arteries or veins, for example. This inadequacy is due to the limitations on resolution and detail from MR imaging of gadolinium agents in the vascular system.

The intimal medial thickness of the innermost two layers of a vessel wall may be measured using ultrasonography. This may be measured using external or intravascular ultrasound (IVUS) methods. However, the usefulness of intimal medial thickness as an indicator of atherosclerosis is disputed. Additionally, changes in the intimal medial thickness over time may also not be correlated with atherosclerosis development, as the systems of vascular response are complex. Further, the differentiation between atherosclerotic plaque and normal thickening of the arteries and veins in particular can be very difficult based on current imaging methods. Current imaging is based on taking a baseline image of a particular patient's vascular system and then comparing that to a later image in order to determine if there has been significant swelling or thickening of the veins or arteries. This thickening could be due to atherosclerotic plaque build up or an immune system response to the atherosclerosis. After the images in the monitored patient indicate thickening, the patient may undergo a more invasive imaging modality involving surgery or intravascular probes. Thus, there are problems with the current methods for diagnosing atherosclerosis. Therefore, the diagnosis of atherosclerosis can not always be made with confidence based on intimal medial thickness measurements alone, and sometimes when the diagnosis of atherosclerosis is felt to be confident, it is incorrect. Additionally, IVUS, like other structural imaging modalities, does not give information about the biology of plaques within the vessel wall.

X-ray, MR, and computed tomography (CT) angiography are likewise inadequate for confident distinction of atheroma or arterial thickening from atherosclerosis in a single imaging evaluation. This is due to also due to the inability to distinguish between normal and abnormal structures indicated, as well as the background noise of blood in the imaged vessels.

Indocyanine green (ICG) may be used in the imaging of atherosclerosis. For example, ICG imaging may be useful for predicting histological types of atheroma. The standard uptake value (SUV) of ICG seen within an atheroma has been shown to be predictive of how aggressive the neovascularization at that site may be. ICG imaging is performed somewhat immediately before or after intravenous administration to the patient due to the short 6 minute half-life of ICG in the bloodstream. However, atheroma and plaques that are seen in the same locations may have variable ICG uptake or binding. Further, where ICG appears to have good sensitivity for binding to atheroma and macrophages, in larger plaques or where endothelial permeability is high, ICG may also accumulate in these interstitial gaps or even leak into intraplaque hemorrhage. Therefore, while the binding profile and accumulation pattern of ICG still highlights the areas of atherosclerotic structures, it may be desirable to have an atherosclerosis imaging agent that exhibits a different or more precise binding profile and that is less subject to first-pass effects associated with short half-lives or circulation times.

From the foregoing, it can be appreciated that there is a need for alternative methods for detecting atheroma in a patient, particularly for the early detection of atherosclerosis before symptoms occur or the onset of advanced atherosclerotic disease. More specifically, there is a need for an imaging agent that may provide a good plaque-to-background ratio, meaning that the targeting imaging agent accumulates in atherosclerotic structures, and not nonspecifically in endothelial, interstitial tissue, or the vessel wall.

### SUMMARY OF THE INVENTION

Quite unexpectedly, the present disclosure has found that methylene blue (MB) has a targeting or binding profile in subjects that is substantially more robust than would be understood or expected by those having skill in the art. Therefore, the present disclosure addresses the foregoing needs by providing methods for the imaging of atherosclerotic structures using MB or related derivatives. MB or related derivatives may accumulate in the atherosclerotic structures such that the progress or risk of atherosclerosis may be diagnosed using various medical imaging techniques, such as planar or tomographic imaging modalities. MB may serve as an atherosclerotic targeting agent for imaging modalities such as angiography, x-ray imaging, computed tomography (CT), magnetic resonance (MR), positron emission tomography (PET), single photon emission tomography, near-infrared spectroscopy (NIRS), fluorescence spectroscopy, fluorescent microscopy (FM), confocal microscopy, high-resolution epifluorescence microscopy, multi-wavelength fluorescence reflectance imaging (FRI), near-infrared fluorescence (NIRF) imaging, optical coherence tomography (OCT), NIRF-OCT, photoacoustic or optoacoustic imaging, ultrasound imaging, intravascular imaging, and any combinations thereof.

The present disclosure provides methods for using MB as a targeting imaging agent for diagnosing atherosclerosis. It has never before been recognized that MB could serve as a diagnostic imaging agent for targeted, molecular, or biological imaging of atherosclerosis. The data laid out in the present disclosure suggests that this is possible using various imaging modalities to detect the binding or uptake pattern of MB. This may allow for an entirely new way to detect atherosclerosis and potentially high-risk plaques in human atherosclerotic vascular disease.

In contrast to Indocyanine green (ICG), MB may be less susceptible to leakage or accumulation in endothelial permeabilities or intraplaque hemorrhaging. Immediately after intravenous administration, ICG rapidly binds to plasma proteins. Further, ICG is removed exclusively by the liver at a rate of 18% to 24% per minute, with a half-life of 150 seconds to about 180 seconds.

The lower leakage susceptibility of MB may be due to the very different binding profile of MB and the fact that the delay time before imaging MB ensures that the blood pool is sufficiently clear and that enough MB uptake has occurred. MB exhibits more complex pharmacokinetics. After intravenous administration, the reduction of MB is multiphasic and includes extensive distribution into deeper compartments and a slow terminal rate of disappearance. The decrease in the urinary excretion rate between 4 hours and 24 hours after administration as well as the estimated terminal phase of 0.0022 minutes indicate MB's half-life at around 5 hours to 6.5 hours. Alternatively, in solutions of radio-iodinated MB the half-life may be about 4.5 hours as reported by measurements of the tracer. Additionally, MB is less amphiphilic than ICG making it less likely to bind with superfluous atherosclerotic structures. Finally, MB shows a different more punctuated binding pattern than ICG with only partial co-localization.

In one aspect, the present disclosure provides a method for using MB as a near-infrared fluorescence imaging agent.

In another aspect, the present disclosure provides a method for diagnosing atherosclerosis in a patient. The method includes administering a solution of methylene blue intravenously to the patient wherein the solution is targeted to any atheroma in the patient. Then an image is acquired to detect the presence of any atheroma in the patient.

In yet another aspect, the present disclosure provides, an imaging method which includes acquiring an image of a human patient suspected of having atherosclerosis and to whom a detectable amount of methylene blue has been administered.

In yet another aspect, the present disclosure provides a compound of formula (I), (II), (Ill), or (IV) below for use in a diagnostic method for detecting atherosclerosis in a patient. The compound of formula (I) is

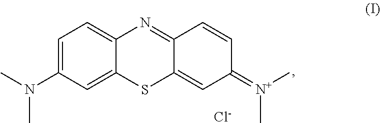

the compound of formula (II) is

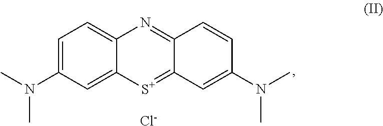

the compound of formula (III) is

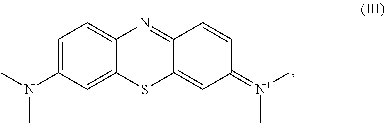

and
the compound of formula (IV) is

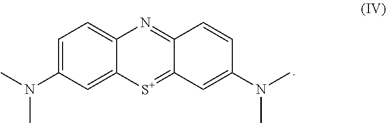

In yet another aspect, the present disclosure provides a method for detecting atherosclerosis in a patient. In this version of the method, a detectable amount of a compound of formula (I), (II), (III) or (IV):

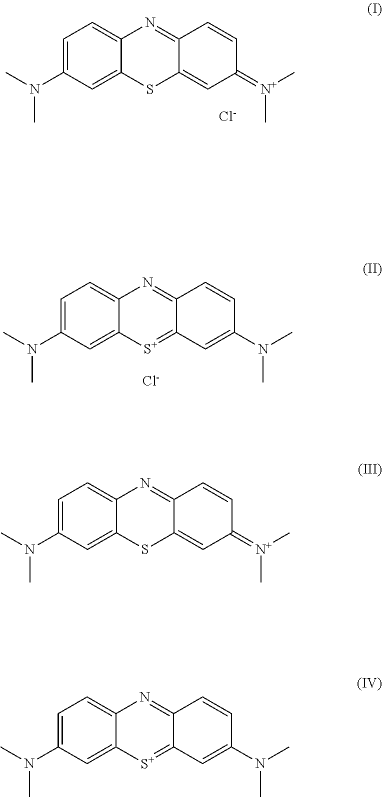

is administered to a patient. The compound is targeted to any atheroma in the patient. An image is then acquired to detect the presence or absence of any atheroma inside veins, arteries, or elsewhere within the patient. The step of acquiring the image can be performed using an imaging method such as angiography, x-ray imaging, computed tomography (CT), magnetic resonance (MR), positron emission tomography (PET), single photon emission tomography, near-infrared spectroscopy (NIRS), fluorescence spectroscopy, fluorescent microscopy (FM), confocal microscopy, high-resolution epifluorescence microscopy, multi-wavelength fluorescence reflectance imaging (FRI), near-infrared fluorescence (NIRF) imaging, optical coherence tomography (OCT), NIRF-OCT, photoacoustic or optoacoustic imaging, ultrasound imaging, intravascular imaging, and any combinations thereof.

In yet another aspect, the present disclosure provides a method for diagnosing atherosclerosis in a patient. The method includes injecting methylene blue into the patient's bloodstream as an indicator targeting atherosclerotic plaque. The method then includes waiting for a number of half-lives until the patient's bloodstream is substantially free of methylene blue. Next, at least a portion of the patient's vascular system is imaged using an imaging modality configured to detect methylene blue that has been bound to atherosclerotic plaque.

These and other features, aspects, and advantages of the present invention will become better understood upon consideration of the following detailed description, drawings, and appended claims.

### BRIEF DESCRIPTION OF THE DRAWINGS

FIG. 1A shows a graph of example measurements of the fluorescence intensity of methylene blue in a bloodstream over time in accordance with the present disclosure.

FIG. 1B shows an example graph of the measured fluorescence intensity of methylene blue over time in accordance with the present disclosure.

FIG. 2 shows an absorption spectrum for methylene blue in accordance with the present disclosure.

FIG. 3A shows a pair of ex vivo images of a set of aortas using multi-wavelength fluorescence reflectance imaging with a white light source, where methylene blue is present in the left image, but not in the right, in accordance with the present disclosure.

FIG. 3B shows a pair of ex vivo images of the set of aortas of FIG. 3A using multi-wavelength fluorescence reflectance imaging with a 470 nm light source, where methylene blue is present in the left image, but not in the right, in accordance with the present disclosure.

FIG. 3C shows a pair of ex vivo images of the set of aortas of FIG. 3A using multi-wavelength fluorescence reflectance imaging with a Cy5 630 nm light source, where methylene blue is present in the left image, but not in the right, in accordance with the present disclosure.

FIG. 3D shows a pair of ex vivo images of the set of aortas of FIG. 3A using multi-wavelength fluorescence reflectance imaging with a Cy7 740 nm light source, where methylene blue is present in the left image, but not in the right, in accordance with the present disclosure.

FIG. 4A shows a graph of the fluorescence intensity of methylene blue in the set of aortas of FIG. 3A as a function of the methylene blue concentration in accordance with the present disclosure.

FIG. 4B shows a chart comparing ranges of target-to-background ratios calculated for the set of aortas of FIG. 3A with and without methylene blue in accordance with the present disclosure.

FIG. 5A shows an ex vivo image of a subject's abdominal aorta after administration of methylene blue in accordance with the present disclosure.

FIG. 5B shows an ex vivo image of the aorta of FIG. 5A using high-resolution epifluorescence microscopy with a white light source in accordance with the present disclosure.

FIG. 5C shows an ex vivo image of the aorta of FIG. 5A using high-resolution epifluorescence microscopy with methylene blue enhanced in accordance with the present disclosure.

FIG. 5D shows a merged image of FIGS. 5B and 5C in accordance with the present disclosure.

FIG. 6A shows a fluorescent microscopy image of a cross-section of a subject's aorta with methylene blue highlighted in white and fluorescein isothiocyanite represented in gray in accordance with the present disclosure.

FIG. 6B shows another example of a fluorescent microscopy image of a cross-section of a subject's aorta with methylene blue highlighted in white and fluorescein isothiocyanite represented in gray in accordance with the present disclosure.

FIG. 7A shows a fluorescent microscopy image of an axial section of a subject's vessel including a venous thrombus with administered methylene blue highlighted in white in accordance with the present disclosure.

FIG. 7B shows a fluorescent microscopy image of the axial section of FIG. 7A with the autofluorescence highlighted in accordance with the present disclosure.

FIG. 7C shows a merged image of FIGS. 7A and 7B in accordance with the present disclosure.

FIG. 8A shows a fluorescent microscopy image of a cross-section of a subject's aorta with methylene blue highlighted in white and fluorescein isothiocyanite represented in gray in accordance with the present disclosure.

FIG. 8B shows an immunohistochemical image of the cross-section of the aorta in FIG. 8A stained with a RAM11 macrophage marker in accordance with the present disclosure.

FIG. 8C shows a histological image of the cross-section of the aorta of FIG. 8A treated with Oil Red 0 stain in accordance with the present disclosure.

FIG. 9A shows a fluorescent microscopy image of a cross-section of a subject's aorta with methylene blue fluorescing in drop-like structures separate from the autofluorescence shown around the edge of the cross-section in accordance with the present disclosure.

FIG. 9B shows an immunohistochemical image of the cross-section of the aorta in FIG. 9A stained with a RAM11 macrophage marker in accordance with the present disclosure.

FIG. 9C shows a histological image of the cross-section of the aorta of FIG. 9A treated with Movat's pentachrome stain in accordance with the present disclosure.

FIG. 9D shows a histological image of the cross-section of the aorta of FIG. 9A stained with hemotoxylin and eosin in accordance with the present disclosure.

FIG. 10 shows images of aortas injected with fluorescein isothiocyanite and methylene blue using multiple modalities including aortic angiography (top), intravascular ultrasound (second from top), and ex vivo multi-wavelength fluorescence reflectance imaging in accordance with the present disclosure.

FIG. 11 shows a fluorescent microscopy image of a representative cross-section of an aorta taken at the location indicated by the vertical white dotted line in the intravascular ultrasound longview image (second from top) in FIG. 10 in accordance with the present disclosure.

FIG. 12 shows a histological image of the aortic cross-section of FIG. 11 stained with of a histology stained with hemotoxylin and eosin in accordance with the present disclosure.

### DETAILED DESCRIPTION OF THE INVENTION

The present disclosure provides methods for the detection of atherosclerosis based on the newly discovered evidence that methylene blue (MB) (or related derivatives) accumulates in areas of atherosclerotic structures, such as within macrophages and atheroma. Based on this discovery, the present disclosure proposes a new use of MB and related derivatives, and a new medical indication for imaging using MB and related derivatives, i.e., the imaging of atheroma to diagnose atherosclerosis without the need for biopsy. Medical imaging using MB and related derivatives may also be used for monitoring the development of atherosclerosis or progress after treatment. Medical imaging using MB and related derivatives may also be useful for diagnosing recurrent/residual atherosclerosis post surgical resection.

The methods in accordance with the present disclosure exploit the newly discovered characteristic of MB or related derivatives to accumulate in areas of atherosclerotic structures. The method of the invention determines the presence (if any) and location of an atherosclerotic structure at a part (e.g., veins or arteries) of the body of a patient as indicated by the presence of MB. The method includes the step of administering a detectable amount of a pharmaceutical composition including MB to a patient. A “detectable amount” means that the amount of the compound that is administered is sufficient to enable detection of accumulation of the compound in an atherosclerotic structure using a medical imaging technique. A “patient” is a mammal, preferably a human, and most preferably a human suspected of developing atherosclerosis. Alternatively, the patient may be a human with no atherosclerotic symptoms, still seeking to rule out or monitor any early stage development of atherosclerosis. MB as used in the present disclosure includes all related derivatives and the various formula compounds described below.

Without intending to be bound by theory, it is believed that MB may be targeting apoptotic, necrotic, or dead cells within atherosclerotic structures in the vascular system of the patient to whom the MB was administered. This apoptosis of lipids, macrophages, or any other part of atherosclerotic plaque is associated with the development of atherosclerosis. MB may function as an apoptosis/necrosis indicator agent for atheroma. MB was first injected as used as a neural imaging agent for targeting mitochondria in 1886. MB may be targeting integral membrane channels of mitochondria between cells. MB may only cross cell boundaries when the cell is apoptotic. Further, viable or live cells may reduce MB leaving the cell unstained. In contrast, unviable or dead cells may remain stained with MB and thus, may be detected through fluorescence, optical, and visible light approaches. Additionally, MB may have molecular targets. Finally, MB may have a unique binding pattern including large, drop-like structures, potentially indicating giant cells, which may be multinucleated or osteoclast-like.

MB may be useful as an imaging agent for diagnosing atherosclerosis because of the cellular uptake of MB in macrophages, which may be markers for high-risk plaques. The present disclosure confirms this MB uptake in example experiments when cross-correlated with immunohistochemical imaging using a RAM11 macrophage marker.

In vivo detection of the accumulated compound MB in the atherosclerotic structures may be achieved by any planar or tomographic medical imaging techniques known or available. MB may serve as an atherosclerotic targeting agent for imaging modalities such as angiography, x-ray imaging, computed tomography (CT), magnetic resonance (MR), positron emission tomography (PET), single photon emission tomography, near-infrared spectroscopy (NIRS), fluorescence spectroscopy, fluorescent microscopy (FM), confocal microscopy, high-resolution epifluorescence microscopy, multi-wavelength fluorescence reflectance imaging (FRI), near-infrared fluorescence (NIRF) imaging, optical coherence tomography (OCT), NIRF-OCT, photoacoustic or optoacoustic imaging, ultrasound imaging, intravascular imaging, and any combinations thereof.

Methylene blue (MB)—also known as basic blue 9, methylthioninium chloride, swiss blue, chromosmon, and urolene blue—has the molecular formulation C₁₆H₁₈ClN₃S. MB is known as a stain or dye for use in histology, but it has never been used or suggested for use in angiography or the detection of atheroma. MB has been used as an optical probe in biophysical systems, as an intercalator in nanoporous materials, as a redox mediator, and in photoelectrochromic imaging. In medical applications, MB inhibits guanylate cyclase and has been used to lower levels of methemoglobin and as a treatment for cyanide poisoning. MB has also been approved by the FDA as an agent for the treatment of globinemia.

Compounds of formulas (I) and (II) below may be used as imaging agents for targeting atherosclerotic regions of interest within a patient.

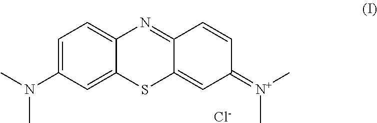

The compound of formula (I) is also known as [7-(dimethylamino)phenothiazin-3-ylidene]-dimethylazanium chloride (CAS Number 61-73-4, 7060-82-4), N-[7-(dimethylamino)-3H-phenothiazin-3-ylidene]-N-methylmethanaminium chloride, or methylene blue (MB). MB has a molecular weight of 319.85 g/mol.

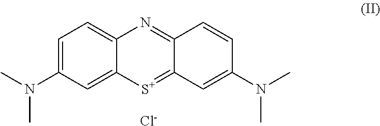

The compound of formula (II) is also known as 7-(Dimethylamino)-N,N-dimethyl-3H-phenothiazin-3-iminium chloride (CAS Number 152071-32-4), 3,7-bis(dimethylamino)phenothiazin-5-ium chloride, methylene blue (MB), or basic blue 9, and has a molecular weight of 319.85 g/mol. MB is a phenothiazine, which is a class of thiazine dyes with similar structures.

Alternatively, a compound of formula (III) or (IV) below may be used as an imaging agent for diagnosing atherosclerosis in a patient.

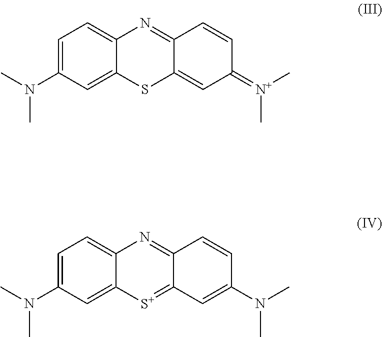

The compound of formula (III) or (IV) is the cation form of formula (I) and (II), respectively. The compound of formula (III) or (IV) may be in solution with a suitable anion, such as phosphate, to stabilize the positive charge of formula (III) or (IV). The compound of formula (III) is also known as [7-(dimethylamino)phenothiazin-3-ylidene]-dimethylazanium, 3,7-bis(dimethylamino)phenothiazin-5-ium, or methylene blue (MB) cation. The compound of formula (IV) is also known as 3,7-Bis(dimethylamino)phenazothionium cation (CAS Number 807306-71-4), 3,7-bis(dimethylamino)phenothiazin-5-ium, or methylene blue (MB) cation. MB and related derivatives usually exist as bromide or chloride salts, but may also be provided in solution with a proper anion to stabilize the positive charge such as phosphate, for example.

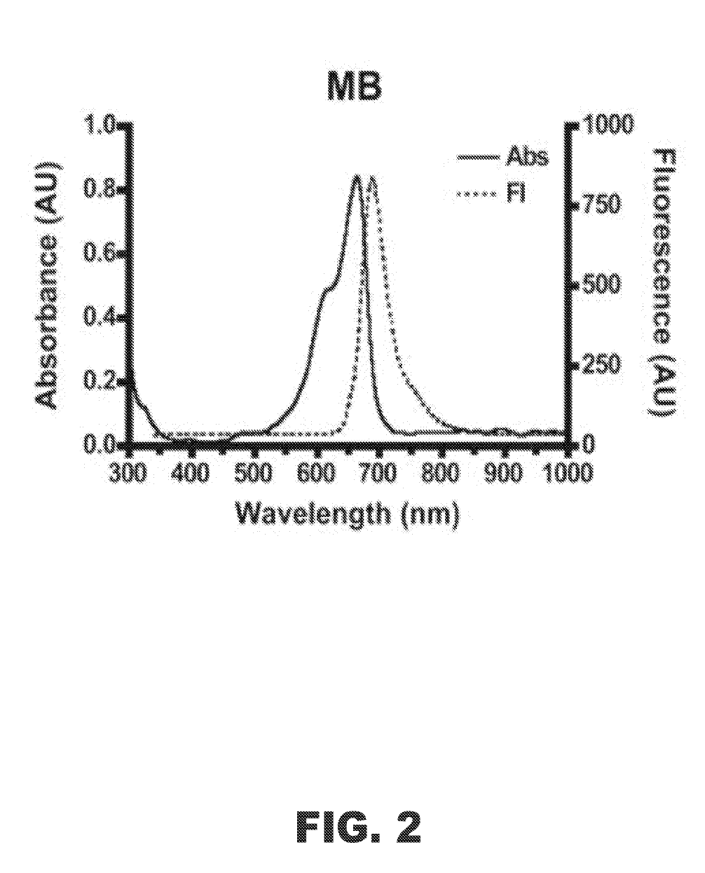

FIG. 2 shows an absorption spectrum for MB, which indicates that MB has near-infrared fluorescence (NIRF). The excitation or absorption wavelength for MB may range from 600 nm to 700 nm. The corresponding fluorescent emission wavelength of MB may range from 650 nm to 720 nm. The quenching threshold for MB in phosphate buffer saline solution is 12.5 μM and the quantum yield is 5.3%. The molar attenuation coefficient of MB in phosphate buffer saline solution is 73,000 cm⁻¹/M. The particular absorption and fluorescent properties of MB depend on the protonation, concentration, and metachromasia of MB and the pH of the MB solution. For example, at higher concentrations (i.e., 31.3 mM) MB may exhibit no NIRF because of quenching and the shift from more monomers to more dimers in solution. In dilute aqueous solutions, spectral studies have shown that the major absorption band at 665 nm may represent monomer absorption, while the shoulder at 610 nm may be due to the dimers. Without intending to be bound by theory, it is believed that the monomer absorption peak of MB at 665 nm corresponds to either the n-π* transition of the n=free doublet of N in the C═N bond and the n=free doublet of S in the S═C bond, or the π-π* transition from S resonating with those from the C's in the thiazinic center. Further, the dimer 610 nm peak at the shoulder may correspond to the 0-1 vibronic transition.

Administration to the patient of a pharmaceutical composition including MB for in vivo detection of the accumulated compound in the atheroma may be accomplished intravenously or intraarterially. Additionally, administration of MB may be intrathecally, intramuscularly, intradermally, subcutaneously, or intracavitary. One non-limiting example method of imaging involves the use of an intravenous injectable molecule such as MB. The administration dosage of MB may vary from 0.1 mg/kg to 15 mg/kg. The MB may be in a solution containing phosphate, water, bromine, chlorine, sulfate, methonal, or other buffers. The buffer solution may be selected based on the desired pH of the resulting MB solution.

In one example method of the present disclosure, sufficient time may be allowed after administration such that the MB may accumulate in any atheroma. This method may therefore include an intentional time delay before imaging. The pharmacokinetics of intravenous administered MB is well established. The time course of MB elimination was measured previously in healthy human volunteers and yielded a blood half-life of 5.25 hours (Peter C et al. Eur J Clin Pharmacol (2000) 56:247-250). Due to the half-lives of certain imaging agents in the bloodstream, vascular imaging may be performed about 3 to 5.5 half-lives after intravenous injection in order to reduce blood-borne background imaging. In one example configuration of the method of the present disclosure, administration of MB into the bloodstream of a patient may occur around 24 hours before image acquisition.

Further, the imaging of MB in the vascular system of the patient may be done with or without a first pass image of an area of interest within the first half-life of MB. This is unlike the imaging procedure required for indocyanine green (ICG) with a half-life of 6 minutes. ICG and other imaging agents with a shorter half-life need a first pass image to be taken immediately before or after intravenous administration in order to acquire a baseline image. The baseline image may be used to overlay images taken after some delay time, such as about a 3 to 5.5 half-life delay, for example, in order to remove the background noise of the imaging agent within the bloodstream and arterial walls.

Particularly when using MB, waiting between 18 and 30 hours after administration to acquire images may allow for MB to clear sufficiently from the patient's blood pool to allow imaging of MB targeting to atheroma. Alternatively, such as when imaging lymph nodes, accumulation of MB may be detectable within 5 to 10 minutes, after which the contrast-to-noise ratio for MB may continue to rise throughout the day. Additionally, this delay time may allow for greater uptake of more fluorophores of MB in the macrophages associated with atheroma.

The different imaging techniques, binding profiles, and cell uptake of MB compared with ICG may be advantageous when using both imaging agents to analyze the same structures within a patient. Cross-correlation or overlays of images taken simultaneously or at different times of MB and ICG within a subject's vascular system may provide more useful diagnostic information than either alone. Alternatively, MB may be combined with another fluorophore besides ICG and used for multi-agent imaging, such as two- or multi-channel near-infrared fluorescence imaging methods.

Advantageously, MB may be used as an imaging agent for confocal microscopy. This is due to MB's fluorescence excitation wavelength being around 600 nm to 700 nm. MB's redder fluorescence absorption range is compatible with the light sources equipped on current confocal microscopes. Additionally, dual-axis confocal microscopy may be used with MB as an in vivo imaging method due to recent advances in the miniaturization of the same.

MB may be used in non-invasive atherosclerosis detection, such as photoacoustic imaging modalities. Photoacoustic or optoacoustic imaging using laser pulses with MB administered intravenously may allow in vivo monitoring of angiogenesis in a patient. With the present disclosure's revelation that the cellular uptake of MB after a delay time has a unique binding profile, MB may be used as a photoacoustic imaging agent that provides enhanced endogenous contrast-to-background ratios.

Atherosclerotic structures may be detected in a patient using non-invasive magnetic resonance (MR) imaging with MB as an intravenous contrast agent. MB may provide higher resolution details of the atherosclerotic structures in the patient because MB may provide unique binding profile and sufficient clearance from the blood pool. Alternatively, MB may be used in black-blood dynamic contrast enhanced MR imaging such that the MB accumulates in the permeable locations of the endothelial tissue of interest or indicates the location and amount of neovessels. This accumulation of MB at certain locations over time may be quantitatively measured.

The targeting imaging retention of MB in the targeted structures allows for better imaging, especially intravascular imaging. One non-limiting example method for imaging atheroma using MB may use near-infrared fluorescence (NIRF) imaging. This method may employ a NIRF catheter to acquire images from a patient's vascular system intravenously. The NIRF catheter may use a continuous wave laser diode with an excitation wavelength in the range of 665 nm to 700 nm as an excitation light source. The excitation light source may be filtered with a narrow band pass interference filter with a 5 nm fullwidth-at-half-maximum (FWHM) in order to remove any residual laser scatter. The filtered excitation light may pass through a beam splitter and then be guided with a multimode fiber. The excitation light source may be coupled into the NIRF catheter. The NIRF catheter may include a radio-opaque tip and a housing. The end of the NIRF catheter may include a prism to direct and focus the light. The NIRF catheter may be advanced into a vein or artery of a patient through a balloon wedge catheter. The NIRF catheter may be manually or automatically pulled back in the veins or arteries of interest while recording a maximum voltage reading. The in vivo plaque target-to-background ratio (TBR) may be calculated as

$$TBR = \frac{{V}_{max}}{{V}_{b}}$$

where Vₘₐₓ is the maximum voltage from all the pullbacks and V_{b} is the background voltage.

### EXAMPLES

The following Examples have been presented in order to further illustrate the invention and are not intended to limit the invention in any way.

### Example 1

Using an atherosclerosis model in rabbits, it has been determined that a routine clinical-type intravenous dose of methylene blue (MB) (1 mg/kg tested) can produce deposition of MB in plaques of rabbits, and can be detected by near-infrared fluorescence (NIRF) imaging.

### Methods:

New Zealand white rabbits (3-4 kg, Charles River Laboratories, n=2) were fed a high cholesterol diet (0.3% total cholesterol, 5% peanut oil; Research Diets) and were subjected to an infrarenal abdominal aorta injury using a 3F Fogarty embolectomy balloon (Edwards Lifesciences). The 3F balloon was inserted percutaneously via the femoral artery, inflated to nominal pressure, and withdrawn under tension and repeated. After recovery from the injury, the rabbits were continued on the 0.3% high cholesterol diet, and their total serum cholesterol levels were routinely measured (Hemagen Diagnostics).

### Methylene Blue:

24 hours prior to atheroma imaging, 1 mg/kg concentration of MB in a phosphate buffer saline solution was administered to the rabbit subjects intravenously.

### In Vivo Imaging:

28 days after aortic injury and continuation on the high cholesterol diet, the rabbits were anesthetized and imaged using various imaging modalities. An aortic angiography (ARCADIS Varic C-arm fluoroscopy unit, Siemens) was performed on the rabbits with a manual contrast injection. After the angiography, an intravascular ultrasound (IVUS) was performed using a catheter (iLab, Boston Scientific). The IVUS catheter was used with an automated 0.5 mm/second pullback across the previously injured aortic region to assess plaque burden. Finally, the rabbit subjects underwent near-infrared fluorescence optical coherence tomography (NIRF-OCT) imaging.

### Ex Vivo Imaging:

Following multimodality imaging, the rabbits were euthanized, and their aortas were removed and placed in saline at 4° C. The freshly resected aortas were imaged using multi-wavelength fluorescence reflectance imaging (FRI) with fluorescein isothiocyanite (FITC) and MB having emission wavelengths of 535 nm and 700 nm, respectively. While using the FRI system (Kodak ImageStation 4000, Carestream Health), the excitation source used for triggering FITC's autofluorescence was focused around 470 nm with an exposure time of 4 seconds. MB's near-infrared fluorescence was triggered by excitation wavelengths around 630 nm for 64 seconds. The regions of interest (ROI) were manually traced with ImageJ (NIH) to circumscribe the atheroma based on a visual assessment of the plaque borders. The atheroma-traced images were then co-registered with the longview IVUS images. The target-to-background ratios (TBRs) were calculated for each ROI as the mean plaque fluorescence divided by the background fluorescence measured in the adjacent normal vessel.

A histology analysis for the aortas was then performed. The excised aortas were filled with an optimal cutting temperature compound and then snap frozen on dry ice. Using a cryostat (Leica CM3050 S), 8 μm sections of the aortas were obtained. The tissue sections were stained for hemotoxylin and eosin (H&E), Masson's Trichrome, and Oil Red 0 (ORO) histological imaging. Additionally, immunohistochemistry tests were performed for macrophages (RAM11; Dako).

Unstained tissue sections were imaged with a multi-wavelength fluorescence microscope (Nikon Eclipse 90i) for FITC and MB. FITC's autofluorescence emissions at 535 nm were excited with a 480 nm source, and MB's near-infrared fluorescent emissions at 700 nm were excited with a 630 nm Cy5.5 source.

### Results:

FIGS. 10-12 show that 28 days after aortic balloon injury and high cholesterol feeding, atheroma were evident as delineated by the angiography, IVUS, and histological imaging.

FIG. 10 shows multiple imaging modalities of the rabbits' aortas including aortic angiography (top), IVUS (second from top), and ex vivo multi-wavelength FRI, with a white light source (middle), 480 nm source (second from bottom), and 630 nm source (bottom). These images demonstrate enhancement of the atheroma by MB—indicated by the white arrowheads in the white light channel image (middle) and the highlighted area of the Cy5.5 image (bottom)—that is distinct from the autofluorescence of FITC (second from bottom). This evidences the unique target binding profile that MB may provide in diagnosing atherosclerosis.

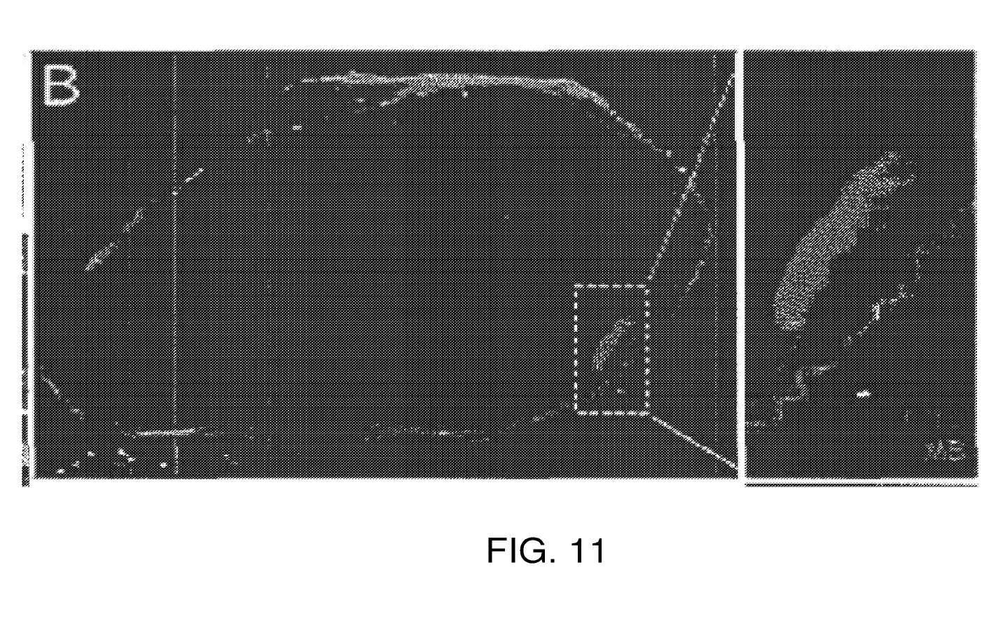

FIG. 11 shows an FM image of a representative cross-section of an aorta taken at the location indicated by the vertical white dotted line in the IVUS longview image (second from top) in FIG. 10. This image reveals that MB is highly targeted to the atheroma as indicated by its near-infrared fluorescence (within dashed line box), which is absent from the normal arterial wall architecture.

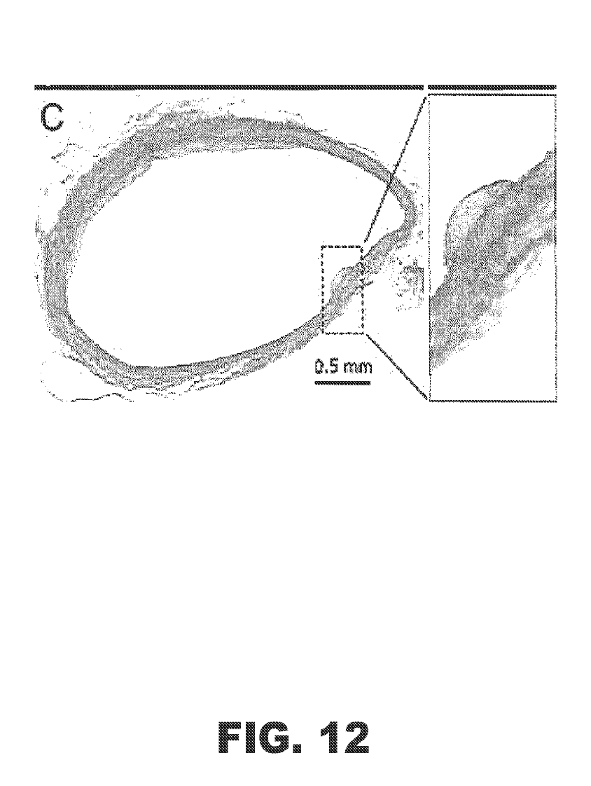

FIG. 12 shows a histological image of the aortic cross-section of FIG. 11 stained with H&E. This H&E stain confirms the presence of atherosclerotic plaque at the site of MB's near-infrared fluorescence enhancement.

This data confirms that atheroma have a unique uptake pattern for MB distinct from any other imaging agents previously known or used.

### Example 2

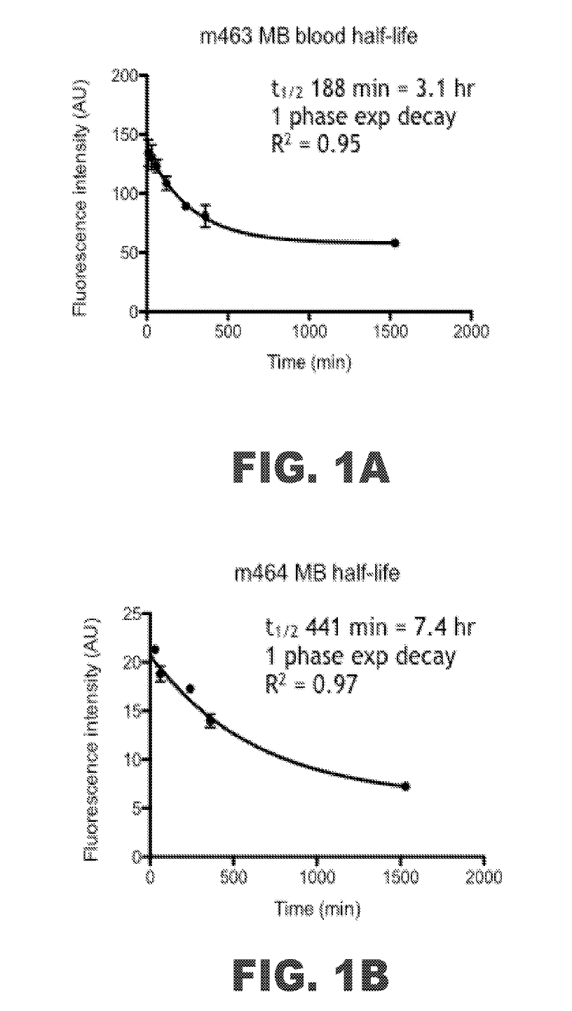

The methylene blue (MB) binding and uptake was evaluated in a rabbit model of atherosclerosis of the abdominal aorta. The half-life for MB in human bloodstreams has been documented as around 5 to 6.5 hours (Peter, Eur. J. Clin. Pharmacol., 2000 56(3):247-50). The half-life for MB in rabbit bloodstreams was measured in two subjects, m463 and m464 that had been subjected to a high cholesterol diet and the high inflammation protocol. FIG. 1A shows a graph of the fluorescence intensity of MB in the bloodstream of m463 over time. The fluorescence intensity had a 1 phase exponential decay. The half-life was found to be 188 minutes, or about 3 hours. FIG. 1B shows a graph of the fluorescence intensity of MB in the bloodstream of m464 over time. The fluorescence intensity similarly had a 1 phase exponential decay. The half-life was found to be 441 minutes, or about 7.4 hours. The average of the MB half-life in the rabbits tested was around 5.25 hours, which falls within the range found in humans.

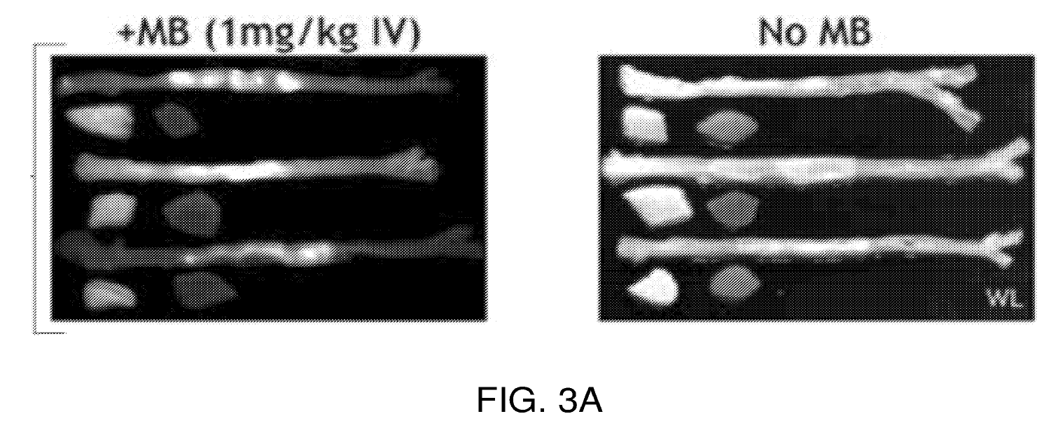

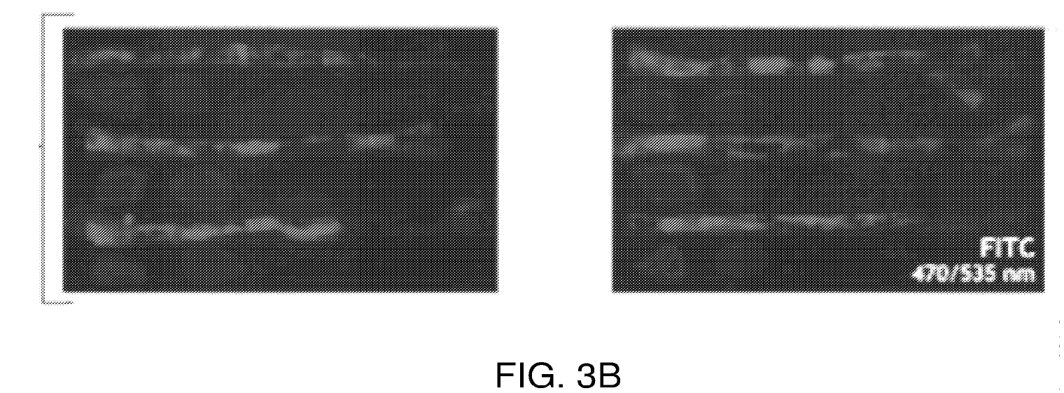

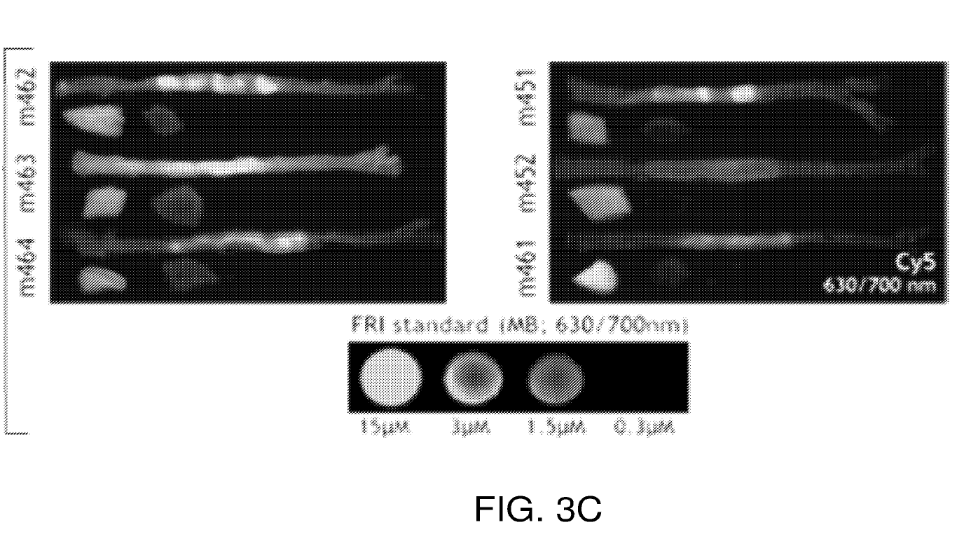

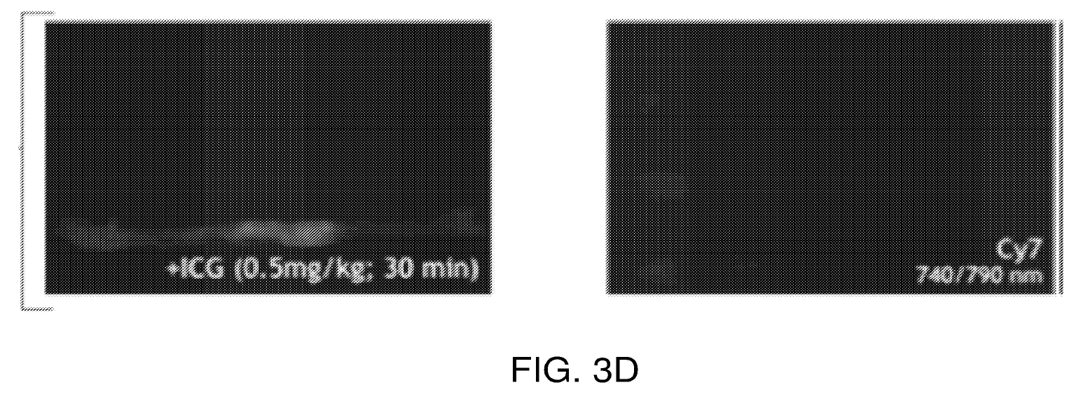

A concentration of 1 mg/kg MB in phosphate buffer saline solution was co-injected with a 0.5 mg/kg solution of indocyanine green (ICG) into the aorta of the rabbit subject, m464, 24 hours and 0.5 hours before imaging, respectively. Fluorescence reflectance imaging (FRI) was performed to evaluate uptake and distribution of MB and ICG. The atherosclerotic zones were outlined in the FRI images as areas of interest. The FRI results showed co-localization of MB and ICG in the atherosclerotic zones of the aorta. For example, FIGS. 3A-3D show multi-wavelength FRI images of the resected aortas of subjects m462 (top), m463 (middle), and m464 (bottom) injected with fluorescein isothiocyanite (FITC), MB, and ICG.

FIG. 3A shows a pair of ex vivo multi-wavelength FRI images of a set of aortas using a white light source with MB present in the left image and no MB present in the right image. The MB present in the left image had been administered to the subject intravenously in a 1 mg/kg phosphate buffer saline solution 24 hours before imaging. FIG. 3B shows a pair of ex vivo images of the aortas of FIG. 3A as seen under a 470 nm excitation light source such that the autofluorescence of FITC at 535 nm may be seen. FIG. 3C shows a pair of ex vivo images of the aortas of FIG. 3A as seen under a Cy5 630 nm excitation light source such that the near-infrared fluorescence of MB around 700 nm may be seen. FIG. 3C further shows an FRI standard scale for MB intensities at 15 μM, 3 μM, 1.5 μM, and 0.3 μM. Finally, FIG. 3D shows a pair of ex vivo images of the set of aortas of FIG. 3A as seen under a Cy7 740 nm excitation light source such that the fluorescence of ICG around 790 nm may be seen. ICG was administered to the subjects intravenously in a 0.5 mg/kg solution 30 minutes before imaging took place.

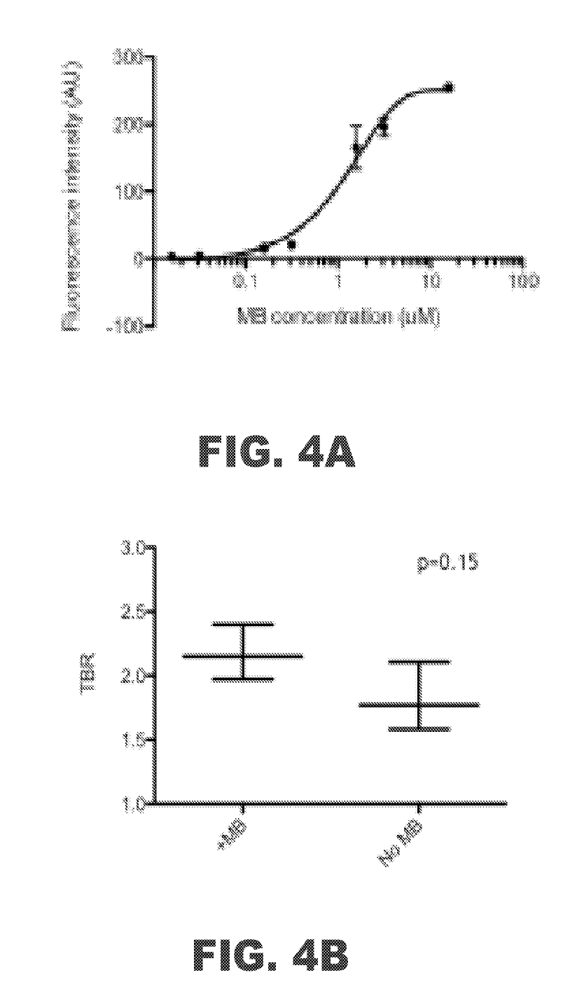

FIG. 4A shows a graph of the fluorescence intensity of MB in the aortas of FIG. 3A as a function of the MB concentration in μM. FIG. 4B shows a chart comparing the ranges of the target-to-background ratios (TBRs) for the aortas of FIG. 3A with MB and without MB. The TBRs were calculated as the mean fluorescence divided by the background fluorescence of the adjacent normal aorta. The comparison of FIG. 4B shows that the aortic imaging of the subjects that were administered MB 24 hours before imaging have higher TBRs than those without MB. This confirms that the targeting profile of MB is substantially more robust than would be expected by those having skill in the art. This higher signal-to-noise ratio is indicative of more MB fluorophore uptake in the atheroma or other atherosclerotic structures as compared to any other imaging agent known or used.

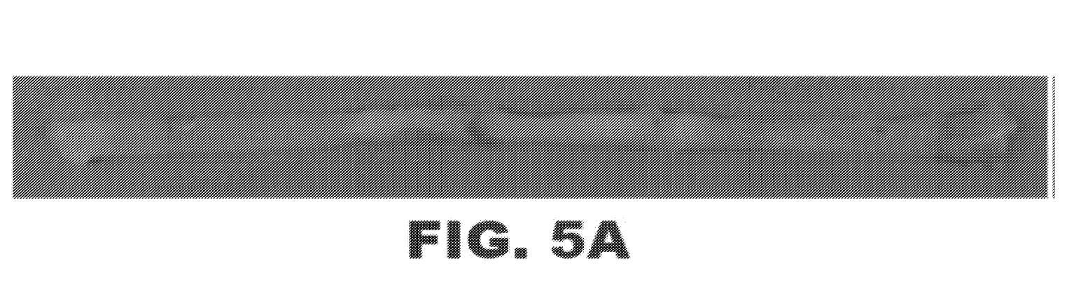

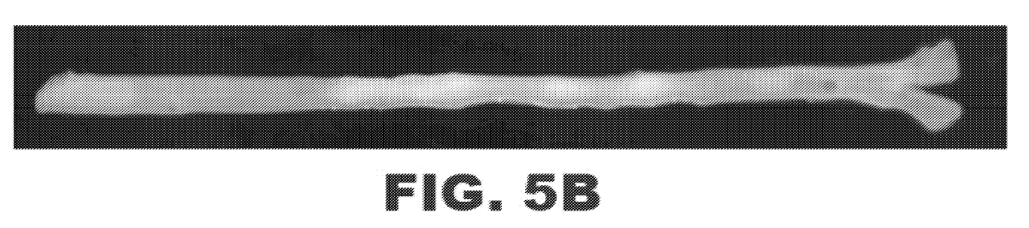

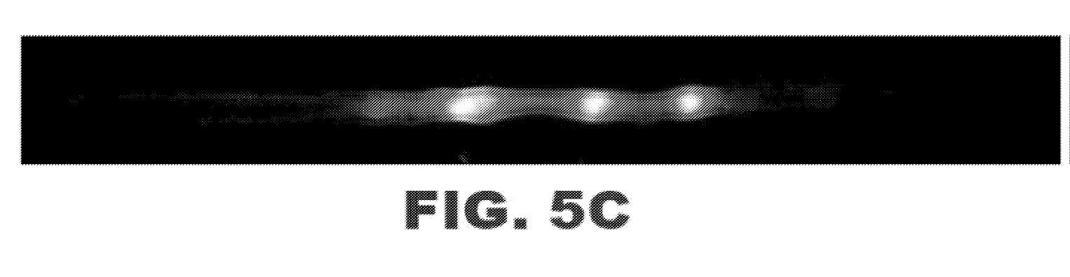

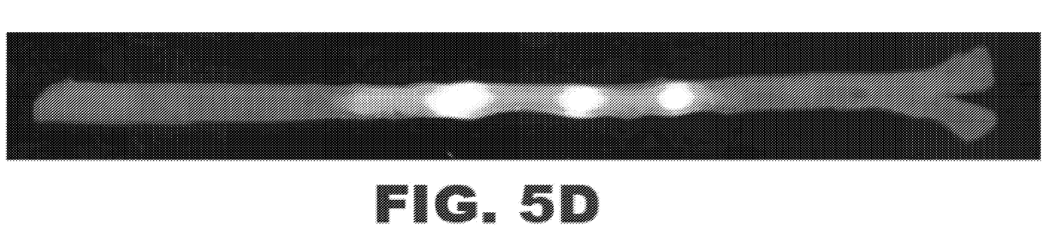

The co-localization of MB and ICG found through FRI was also verified using high-resolution epifluorescence microscopy of fresh-frozen tissue cross-sections from the areas of interest outlined in the FRI results. MB uptake was found to localize in both microscopic lesions and atherosclerotic lesions with greater than 60% stenosis. Further, the MB binding was associated with cellular uptake when evaluating sections counterstained with a fluorescent nuclear stain. For example, FIG. 5A shows an image of a resected abdominal aorta of a subject on a high cholesterol diet and high inflammation protocol after administration of MB. The administration of MB in the subject Rb m539 as seen in FIGS. 5A-5D was 24 hours before imaging took place and included a 1 mg/kg solution of MB in a phosphate buffer saline solution. FIG. 5B shows an ex vivo image of the aorta of FIG. 5A using high-resolution epifluorescence microscopy with a white light source. FIG. 5C shows an ex vivo image of the aorta of FIG. 5A using high-resolution epifluorescence microscopy with MB enhanced. FIG. 5D shows a merged image of FIGS. 5B and 5C.

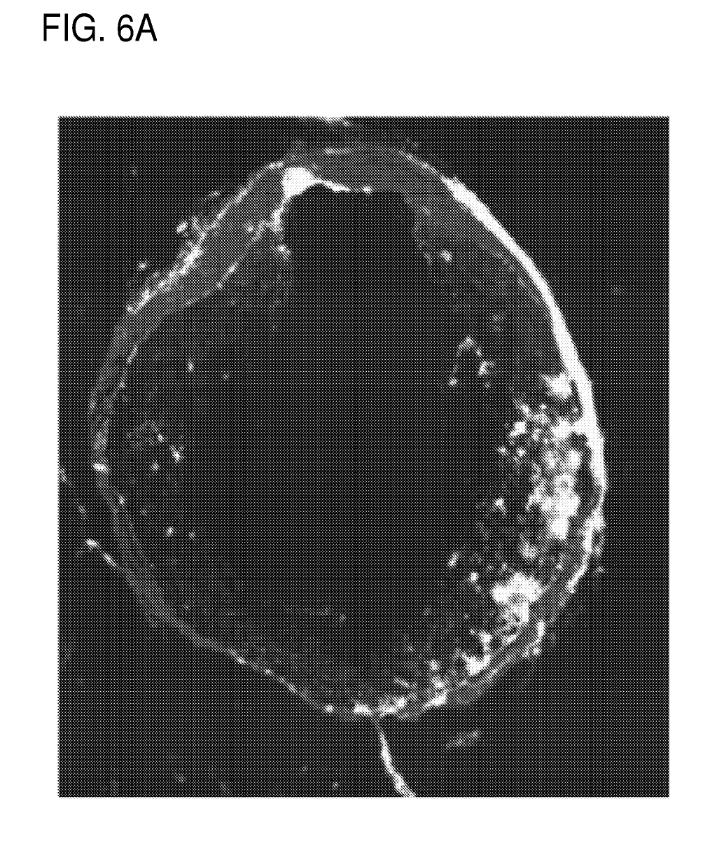

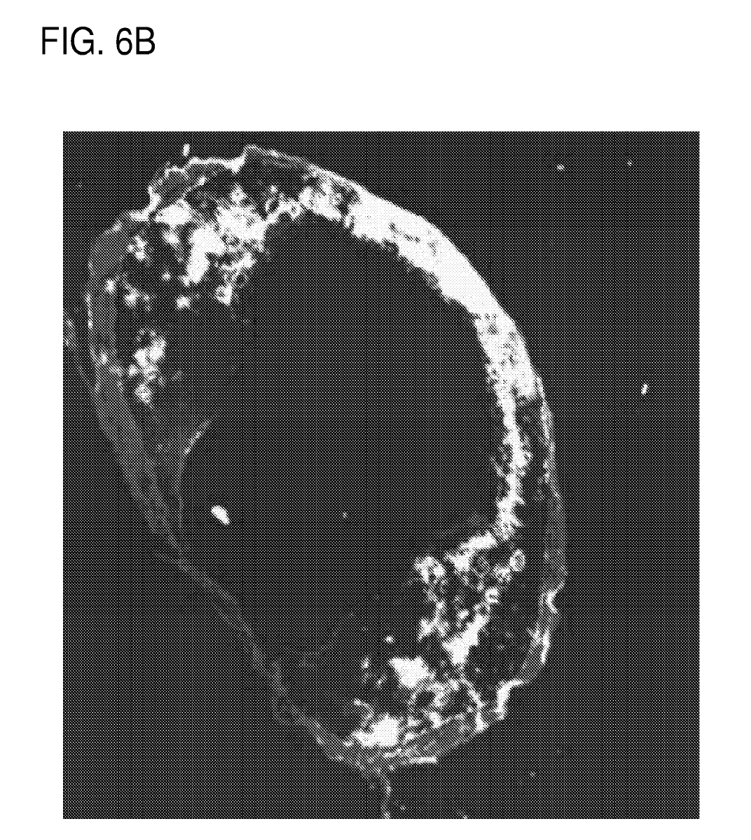

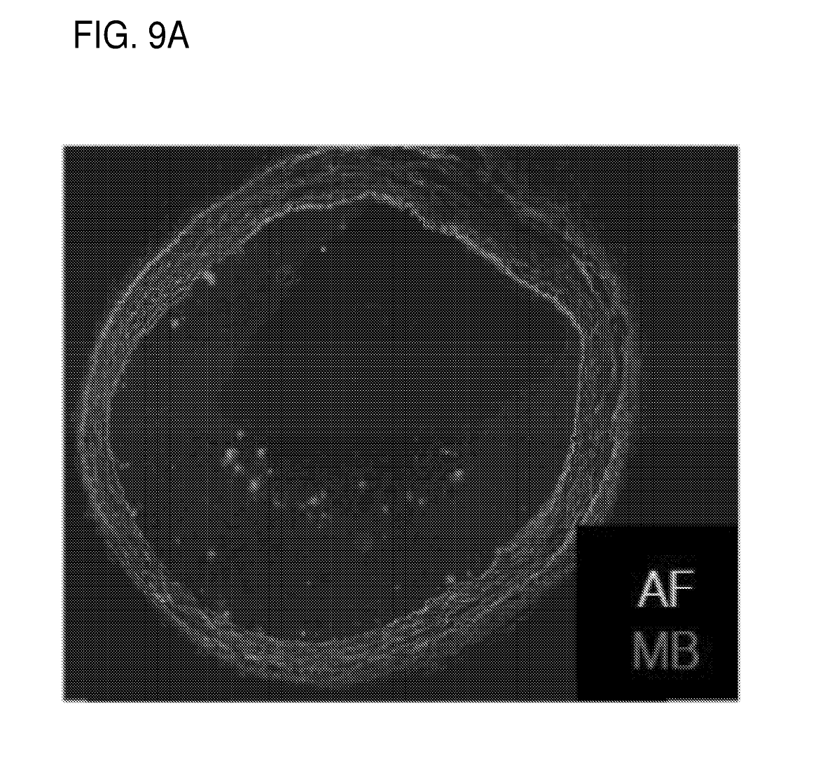

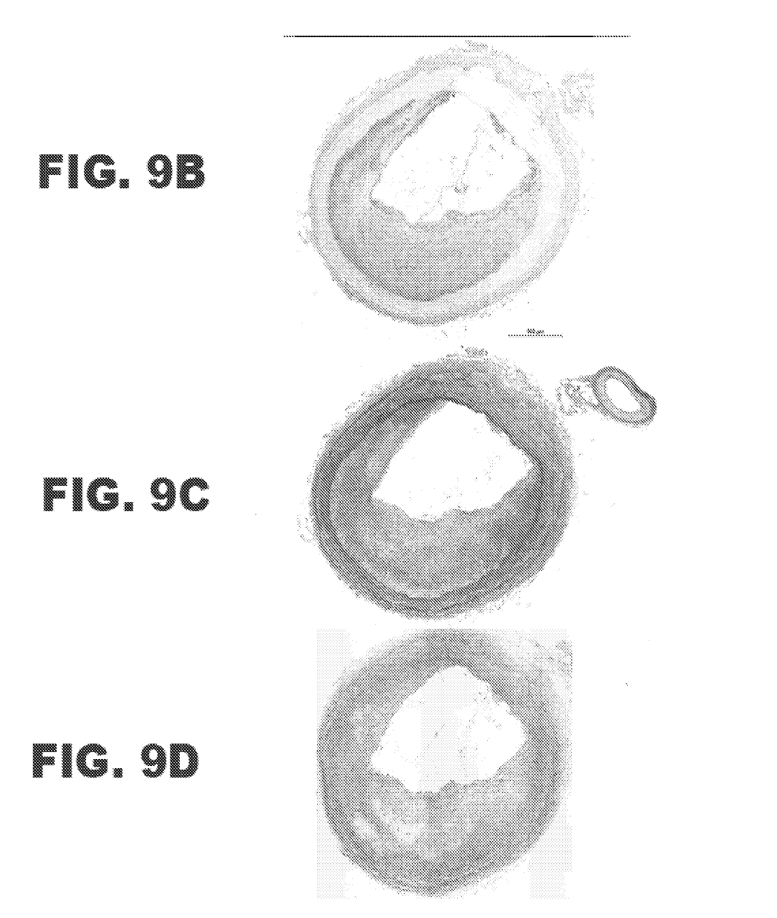

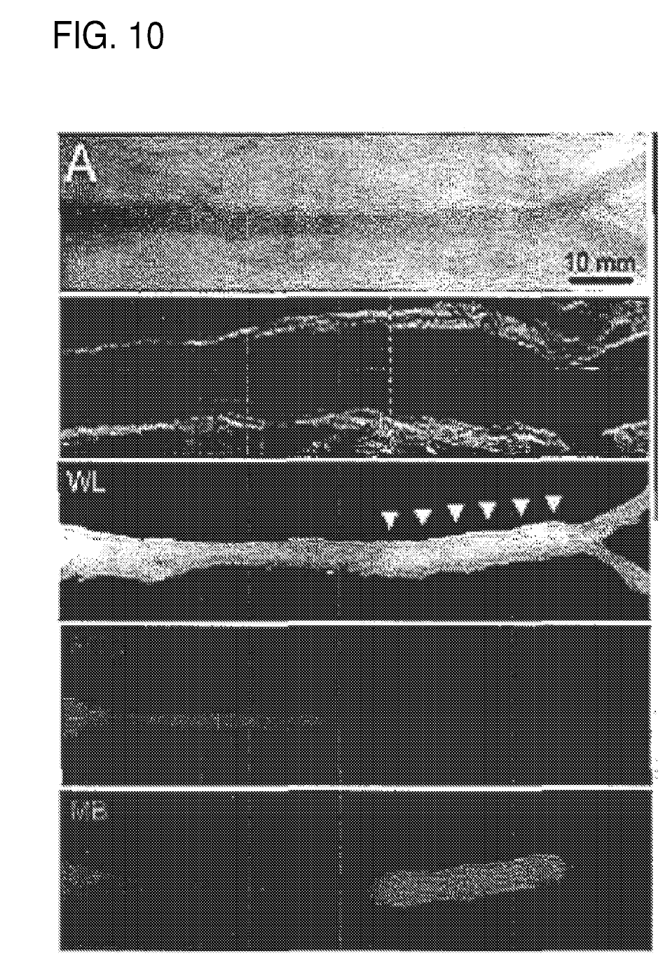

Fluorescent microscopy (FM) was performed on a cross-section of the aorta of m464 in the fluorescein isothiocyanite (FITC) and Cy5 channels. FIG. 6A shows an FM image (10× Stitch on 90i) of a cross-section of the aorta of subject m464 with MB and FITC present. The white areas represent the near-infrared fluorescence of MB under a Cy5.5 source. The grey areas represent the autofluorescence of FITC. There is little overlap between the MB and FITC areas, evidencing the unique uptake and binding profile for MB in atherosclerotic structures not targeted by other imaging agents. FIG. 6B shows another example of an FM image (10× Stitch on 90i) of a cross-section of the aorta of subject m464 with MB and FITC present. Again, the distinction between the areas of MB binding and FITC autofluorescence indicate MB's utility as an atheroma targeting agent unlike any other. Further, the TBR of MB in FIGS. 6A and 6B illustrates the robustness of the targeting profile of MB. In yet another example, FIG. 9A shows an FM image of a cross-section of the aorta of subject m539 after having been fed a high cholesterol diet for 28 days after undergoing aortic injury according to the high inflammation protocol. The FM image shows autofluorescence around the aortic walls and the near-infrared fluorescence of MB in distinct spots or drops along the inner aortic walls, separate from the autofluorescence.

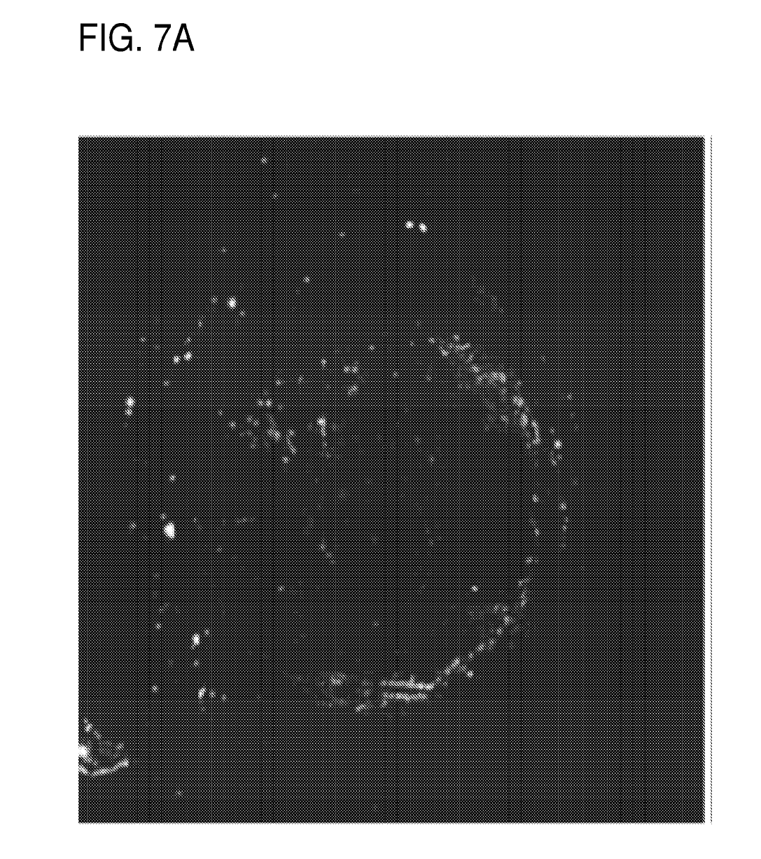

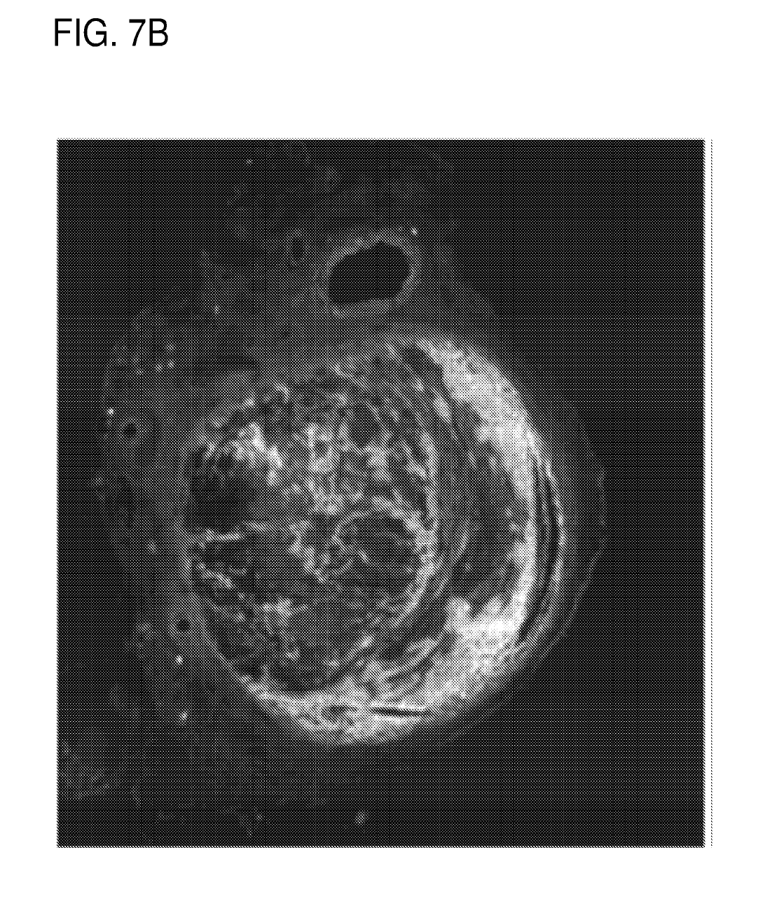

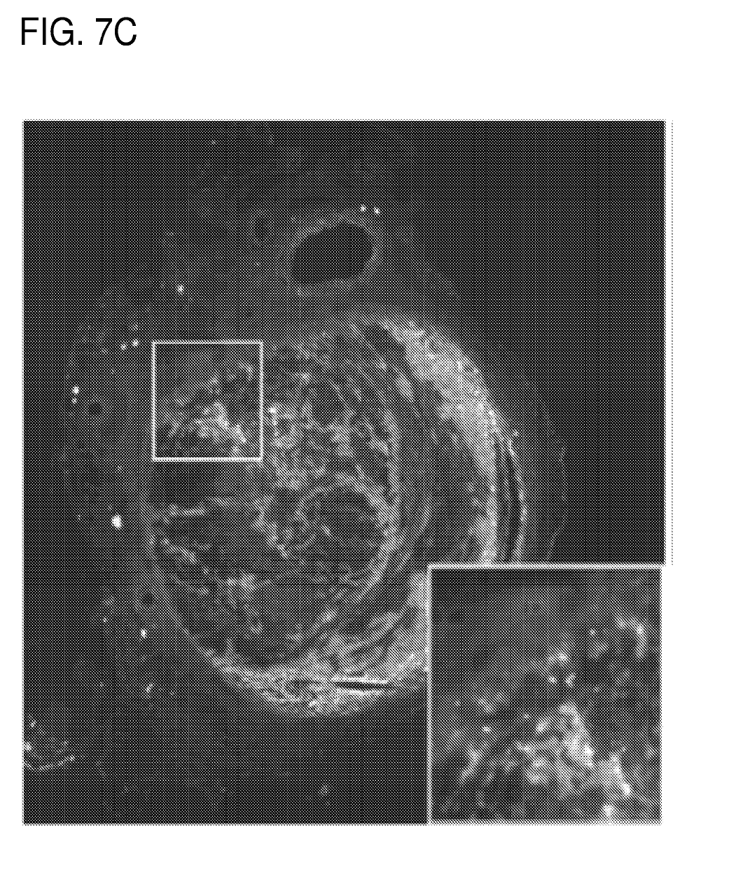

Moreover, FIGS. 7A-7C show FM images of an axial section of a subject's vessel including a venous thrombus after undergoing vessel injury according to the high inflammation protocol. The subject was injected with a 1 mg/kg MB concentration in a phosphate buffer saline solution 24 hours prior to imaging. FIG. 7A shows an FM image of the axial section including the venous thrombus acquired using a filter so that just the MB is highlighted. FIG. 7B shows an FM image of the same axial section in FIG. 7A with a filter applied such that only the autofluorescence of the other imaging agent is highlighted. Finally, FIG. 7C shows a merged image of FIGS. 7A and 7B where MB is highlighted in white and the autofluorescence of the other imaging agent is represented in a darker shade. The boxed area in FIG. 7C shows the venous thrombus in the vessel. The enlargement of the venous thrombus portion in the corner of FIG. 7C shows the areas of specific MB uptake that are not shown in the autofluorescence. The FM images of FIGS. 7A-7C show autofluorescence around the vessel walls and the near-infrared fluorescence of MB in distinct spots or drops along the inner vessel walls and in the venous thrombus, separate from the autofluorescence. This again evidences the unique binding pattern that MB may offer for vascular imaging.

MB binding in the aortas of the subjects was also evaluated using traditional histological stains. A tissue cross-section fluorescently imaged to evaluate MB uptake was further treated with a hemotoxylin and eosin (H&E) stain. The histological imaging of the cross-section showed that the areas of MB binding were not limited to superficial areas of atherosclerotic lesions. In particular, MB binding was seen in specific regions permeating the entire depth of the lesions. Even less cellular areas were also found to have MB uptake. For example, FIG. 9D shows a histological image of the cross-section of the aorta of FIG. 9A stained with H&E. Additionally, FIG. 9C shows a histological image of the cross-section of the aorta of FIG. 9A treated with Movat's pentachrome stain.

Finally, the cross-section was imaged using immunohistochemical-staining techniques. The immunohistochemical stain RAM11 which is a known macrophage marker was used to cross-correlate the MB binding images. The areas of MB uptake were found to be associated with areas positive for macrophages in the RAM11 staining. These areas where not limited to a specific location. For example, one area of consistent MB and RAM11 co-localization was located near the luminal side of internal elastic lamina as well as other regions of the atheroma. FIG. 9B shows an immunohistochemical image of the cross-section of the aorta in FIG. 9A stained with the RAM11 macrophage marker.

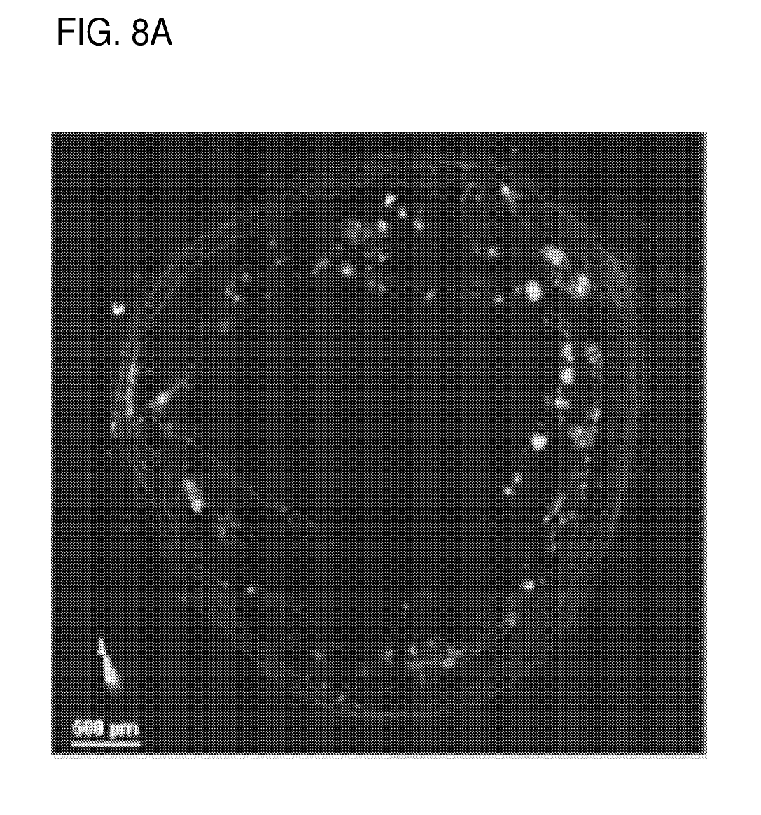

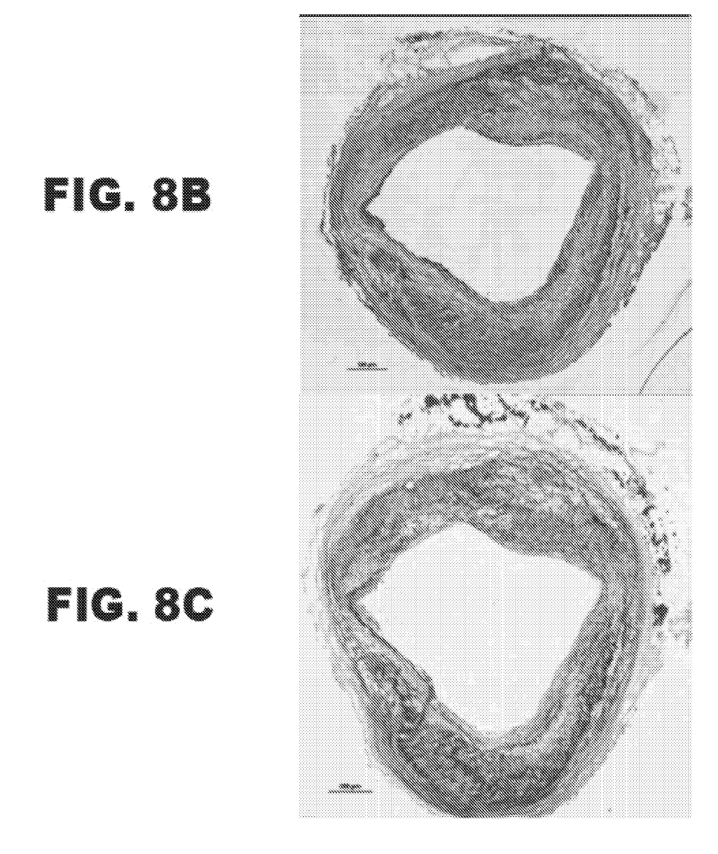

As another example of this co-localization between MB and RAM11, FIGS. 8A-8C show images of the same aortic cross-section of a subject using various imaging modalities, including FM, histological imaging, immunohistochemical imaging. FIG. 8A shows an FM image of a cross-section of a subject's aorta with the near-infrared fluorescence of MB in distinct white spots or drops along the inner aortic walls, separate from the autofluorescence of FITC represented in gray along the normal arterial wall architecture. FIG. 8B shows an immunohistochemical image of the cross-section of the aorta in FIG. 8A stained with a RAM11 macrophage marker. FIG. 8C shows a histological image of the cross-section of the aorta of FIG. 8A treated with Oil Red 0 (ORO) stain.

### CONCLUSIONS

The foregoing has shown that MB has the potential to become a useful targeted imaging agent for the diagnosis of atherosclerosis. Further, MB may help resolve common diagnostic dilemmas in atherosclerosis diagnosis. Atheroma imaging with MB represents a new type of atherosclerotic imaging not previously described. The exact mechanism of MB binding in meningiomas is not clear, but MB may be binding to apoptotic cells within atheroma. This binding is unique from ICG and other known imaging agents. MB may show a characteristic shape of large, drop-like structures. Further, MB may be permeating integral membrane channels.

Although the present invention has been described in detail with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

1. A method for diagnosing atherosclerosis in a patient, the method comprising:
(a) administering a solution of methylene blue intravenously to the patient wherein the solution is targeted to any atheroma in the patient; and
(b) acquiring an image to detect the presence of any atheroma in the patient.

2. The method of claim 1, wherein the solution includes methylene blue at a concentration of 0.1 to 5 mg/kg.

3. The method of claim 2, wherein the solution includes methylene blue at a concentration of 0.2 to 2 mg/kg.

4. The method of claim 2, wherein the solution includes methylene blue at a concentration of 0.5 to 1.5 mg/kg.

5. The method of claim 1, wherein the solution includes phosphate buffer saline.

6. The method of claim 1, wherein acquiring the image takes place after waiting for a delay time after administering the solution to the patient.

7. The method of claim 6, wherein the solution does not bind with the normal endothelial walls in a vessel of the patient after the delay time.

8. The method of claim 6, wherein the delay time is between about 18 hours and about 33 hours.

9. The method of claim 1, wherein the solution is targeted to macrophage cells within the atheroma.

10. The method of claim 1, wherein the solution is targeted to apoptotic cells within the atheroma.

11. The method of claim 1, wherein acquiring the image comprises using an in vivo imaging method.

12. The method of claim 1, wherein acquiring the image comprises using a non-invasive imaging method.

13. The method of claim 1, wherein acquiring the image comprises using an angiographic imaging modality wherein the solution acts as a contrast agent.

14. The method of claim 13, wherein the angiographic imaging modality is x-ray imaging.

15. The method of claim 13, wherein the angiographic imaging modality is magnetic resonance imaging.

16. The method of claim 1, wherein acquiring the image comprises using a tomographic imaging modality.

17. The method of claim 16, wherein the tomographic imaging modality is computed tomography.

18. The method of claim 16, wherein the tomographic imaging modality is positron emission tomography.

19. The method of claim 16, wherein the tomographic imaging modality is single photon emission tomography.

20. The method of claim 16, wherein the tomographic imaging modality is optical coherence tomography.

21. The method of claim 16, wherein the tomographic imaging modality is near-infrared fluorescence optical coherence tomography.

22. The method of claim 1, wherein acquiring the image comprises using near-infrared spectroscopy.

23. The method of claim 1, wherein acquiring the image comprises using fluorescent microscopy.

24. The method of claim 1, wherein acquiring the image comprises using confocal microscopy.

25. The method of claim 1, wherein acquiring the image comprises using high-resolution epifluorescence microscopy.

26. The method of claim 1, wherein acquiring the image comprises using multi-wavelength fluorescence reflectance imaging.

27. The method of claim 1, wherein acquiring the image comprises using near-infrared fluorescence imaging.

28. The method of claim 1, wherein acquiring the image comprises using photoacoustic imaging.

29. The method of claim 1, wherein acquiring the image comprises using an invasive imaging method.

30. The method of claim 1, wherein acquiring the image comprises using intravascular imaging.

31. The method of claim 1, wherein acquiring the image comprises using fluorescent imaging.

32. The method of claim 1, wherein acquiring the image comprises using fluorescence spectroscopy.

33. The method of claim 1, wherein acquiring the image comprises using a near-infrared fluorescence probe.

34. The method of claim 1, further comprising acquiring a second image using a second imaging modality.

35. The method of claim 34, wherein the second imaging modality is intravascular ultrasound imaging.

36. The method of claim 24, further comprising cross-correlating the image and the second image.

37. The method of claim 1, wherein the presence of any atheroma in the patient is indicated by greater activity of the solution.

38. The method of claim 1, wherein the presence of any atheroma in the patient is indicated by fluorescent emissions in particular regions where the atheroma are located.

39. The method of claim 38, wherein the fluorescent emissions are between about 650 nm and about 720 nm.

40.-78. (canceled)

79. A method for diagnosing atherosclerosis in a patient, the method comprising:
(a) administering intravenously to the patient a solution containing a detectable amount of a compound of formula (I), (II), (III), or (IV): 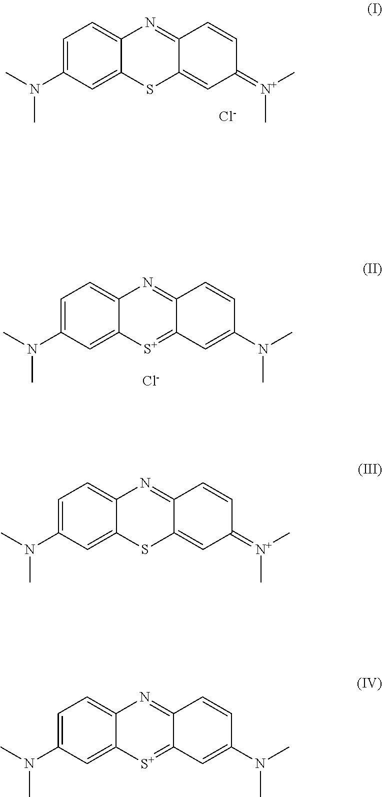
wherein the compound is targeted to any atheroma in the patient; and
(b) acquiring an image to detect the presence of any atheroma in the patient.

80. The method of claim 79, wherein acquiring the image includes using at least one of angiography, x-ray imaging, computed tomography (CT), magnetic resonance (MR), positron emission tomography (PET), single photon emission tomography, near-infrared spectroscopy (NIRS), fluorescence spectroscopy, fluorescent microscopy (FM), confocal microscopy, high-resolution epifluorescence microscopy, multi-wavelength fluorescence reflectance imaging (FM), near-infrared fluorescence (NIRF) imaging, optical coherence tomography (OCT), NIRF-OCT, photoacoustic or optoacoustic imaging, ultrasound imaging, and intravascular imaging.

81. A method for diagnosing atherosclerosis in a patient, the method comprising:
a. injecting methylene blue (MB) into the patient's bloodstream as an indicator targeting atherosclerotic plaque;
b. waiting for a number of half-lives until the patient's bloodstream is substantially free of MB;
c. imaging at least a portion of the patient's vascular system using an imaging modality configured to detect MB bound to atherosclerotic plaque.

82. The method of claim 81, wherein the number of half-lives is between about 3 and about 5.5.

83. The method of claim 81, wherein the imaging modality is at least one of angiography, x-ray imaging, computed tomography (CT), magnetic resonance (MR), positron emission tomography (PET), single photon emission tomography, near-infrared spectroscopy (NIRS), fluorescence spectroscopy, fluorescent microscopy (FM), confocal microscopy, high-resolution epifluorescence microscopy, multi-wavelength fluorescence reflectance imaging (FRI), near-infrared fluorescence (NIRF) imaging, optical coherence tomography (OCT), NIRF-OCT, photoacoustic or optoacoustic imaging, ultrasound imaging, and intravascular imaging.
